(19) United States
(12) Patent Application Publication
(10) Pub. No.: US 2008/0015627 A1
(43) Pub. Date: Jan. 17, 2008

(54) REMOVABLE ANCHORED LUNG VOLUME REDUCTION DEVICES AND METHODS

(76) Inventor: Lauri J. DeVore, Seattle, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/781,130

(22) Filed: Jul. 20, 2007

Related U.S. Application Data
(63) Continuation of application No. 10/124,790, filed on Apr. 16, 2002, now abandoned.

(51) Int. Cl. A61M 16/00

(52) U.S. Cl. 606/192; 606/198

(57) ABSTRACT

An intra-bronchial device may be placed in an air passageway of a patient to collapse a lung portion associated with the air passageway. The device includes an obstructing member that prevents air from being inhaled into the lung portion to collapse the lung portion, and an anchoring device that anchors the obstructing member in the air passageway by engaging the obstructing member and the air passageway wall. The anchoring device may frictionally engage the obstructing member and the air passageway, or engage both by piercing. The engagement provided by the anchoring device may be releasable for removal of the obstructing member. The anchoring device may be balloon expandable from a first shape to a second shape that engages the obstructing member and the air passageway. The obstructing member may be a one-way valve.

### RELATED APPLICATIONS

The present application is a continuation of U.S. application Ser. No. 10/124,790 filed Apr. 16, 2002, the entire contents of which is incorporated herein by reference.

### BACKGROUND

The present invention is generally directed to a removable anchored device, system, and method for treating Chronic Obstructive Pulmonary Disease (COPD). The present invention is more particularly directed to providing an anchored intra-bronchial obstruction that may be removable.

COPD has become a major cause of morbidity and mortality in the United States over the last three decades. COPD is characterized by the presence of airflow obstruction due to chronic bronchitis or emphysema. The airflow obstruction in COPD is due largely to structural abnormalities in the smaller airways. Important causes are inflammation, fibrosis, goblet cell metaplasia, and smooth muscle hypertrophy in terminal bronchioles.

The incidence, prevalence, and health-related costs of COPD are on the rise. Mortality due to COPD is also on the rise. In 1991, COPD was the fourth leading cause of death in the United States and had increased 33% since 1979. COPD affects the patient's whole life, producing increasing disability. It has three main symptoms: cough; breathlessness; and wheeze. At first, breathlessness may be noticed when running for a bus, digging in the garden, or walking uphill. Later, it may be noticed when simply walking in the kitchen. Over time, it may occur with less and less effort until it is present all of the time. COPD is a progressive disease and currently has no cure. Current treatments for COPD include the prevention of further respiratory damage, pharmacotherapy, and surgery. Each is discussed below.

The prevention of further respiratory damage entails the adoption of a healthy lifestyle. Smoking cessation is believed to be the single most important therapeutic intervention. However, regular exercise and weight control are also important. Patients whose symptoms restrict their daily activities or who otherwise have an impaired quality of life may require a pulmonary rehabilitation program including ventilatory muscle training and breathing retraining. Long-term oxygen therapy may also become necessary.

Pharmacotherapy may include bronchodilator therapy to open up the airways as much as possible or inhaled beta-agonists. For those patients who respond poorly to the foregoing or who have persistent symptoms, ipratropium bromide may be indicated. Further, courses of steroids, such as corticosteroids, may be required. Lastly, antibiotics may be required to prevent infections and influenza and pneumococcal vaccines may be routinely administered. Unfortunately, there is no evidence that early, regular use of pharmacotherapy will alter the progression of COPD.

About 40 years ago, it was first postulated that the tethering force that tends to keep the intratharacic airways open was lost in emphysema and that by surgically removing the most affected parts of the lungs, the force could be partially restored. Although the surgery was deemed promising, the lung volume reduction surgery (LVRS) procedure was abandoned. LVRS was later revived. In the early 1990's, hundreds of patients underwent the procedure. However, the number of procedures declined because Medicare stopping reimbursing for LVRS. The procedure is currently under review in controlled clinical trials. However, preliminary data indicates that patients benefit from the procedure in terms of an increase in forced expiratory volume, a decrease in total lung capacity, and a significant improvement in lung function, dyspnea, and quality of life. Improvements in pulmonary function after LVRS have been attributed to at least four possible mechanisms; enhanced elastic lung recoil, correction of ventilation/perfusion mismatch, improved efficiency of respiratory musculature, and improved right ventricular filling.

Lastly, lung transplantation is also a therapeutic option. Today, COPD is the most common diagnosis for which lung transplantation is considered. Unfortunately, this consideration is given for only those with advanced COPD. Given the limited availability of donor organs, lung transplant is far from being available to all patients.

There is a need for additional non-surgical options for permanently treating COPD without surgery. A promising new therapy includes non-surgical apparatus and procedures for lung volume reduction by permanently obstructing the air passageway that communicates with the portion of the lung to be collapsed. The therapy includes placing an obstruction in the air passageway that prevents inhaled air from flowing into the portion of the lung to be collapsed. This provides lung volume reduction with concomitant improved pulmonary function without the need for surgery. The effectiveness of obstructions may be enhanced if it is anchored in place. The effectiveness may also be enhanced if the obstruction is removable. However, no readily available apparatus and method exists for anchoring the obstruction, and for removal if required.

In view of the foregoing, there is a need in the art for a new and improved apparatus and method for permanently obstructing an air passageway that is anchored in place, and that may be removed if required. The present invention is directed to a device, system, and method that provide such an improved apparatus and method for treating COPD.

### SUMMARY

The present invention provides an intra-bronchial device for placement in an air passageway of a patient to collapse a lung portion associated with the air passageway. The device includes an obstructing member that prevents air from being inhaled into the lung portion to collapse the lung portion, and an anchoring device that anchors the obstructing member in the air passageway by engaging the obstructing member and the air passageway wall. The anchoring device may frictionally engage the obstructing member. The engagement provided by the anchoring device may be releasable for removal of the obstructing member. The anchoring device may comprise a material having a memory of an original undistorted shape, and a resiliency to return the material from a distorted shape to the original undistorted shape. The anchoring device may be balloon expandable from a first shape to a second shape that engages the obstructing member and the air passageway. The obstructing member may be a one-way valve.

An alternative embodiment of the present invention provides an intra-bronchial device for placement in an air passageway of a patient to collapse a lung portion associated with the air passageway. The device includes an obstructing member that prevents air from being inhaled into the lung portion to collapse the lung portion, and an anchoring device having a projection that anchors the obstructing member in the air passageway by piercingly engaging the obstructing member and the air passageway wall. The engagement provided by the anchoring device may be releasable for removal of the obstructing member. The anchoring device may comprise a material having a memory of an original undistorted shape, and a resiliency to return the material from a distorted shape to the original undistorted shape. The anchoring device may be balloon expandable from a compressed shape to a deployed shape that engages the obstructing member and the air passageway wall. The anchoring device may be configured to urge engagement with the air passageway wall. The projection may be releasable from the air passageway wall for removal of the anchoring device. The projection may include a stop dimensioned to limit the piercing. At least a portion of the anchoring device may be collapsible for placement in the air passageway. The anchoring device may collapse centrally. The anchoring device may include a projection that collapses centrally. The anchoring device may be configured to move from a first position to a second position to anchor the obstructing member in the air passageway. The anchoring device may be configured to move from a first position to a second position to anchor the obstructing member in the air passageway, and to move from the second position to the first position to disengage the obstructing member for removal from the air passageway. The obstructing member may be a one-way valve.

Another alternative embodiment provides a method of reducing the size of a lung by collapsing a portion of the lung. The method includes the step of providing an intra-bronchial device having an obstructing member which is so dimensioned when deployed in an air passageway communicating with the portion of the lung to be collapsed to preclude air from being inhaled, and an anchoring device that anchors the obstructing member in the air passageway by engaging the obstructing member and the wall of the air passageway. The method also includes the steps of placing the obstructing member in the air passageway, placing the anchoring device in the air passageway, and deploying the anchoring device. The anchoring device may include a projection that piercingly engages the obstructing member and the air 3 passageway wall. The anchoring device may be releasable for removal of the intra-bronchial device. The obstructing member may form a one-way valve. At least a portion of the anchoring device may be collapsible.

A further embodiment provides a method of reducing the size of a lung by collapsing a portion of the lung. The method includes the step of providing an intra-bronchial device having an obstructing member which is so dimensioned when deployed in an air passageway communicating with the portion of the lung to be collapsed to preclude air from being inhaled, and an anchoring device that anchors the obstructing member in the air passageway by engaging the obstructing member and the wall of the air passageway. The method also includes the steps of placing the obstructing member in the air passageway, placing the anchoring device in the air passageway, deploying the anchoring device, removing the anchoring device, and removing the obstructing member. The anchoring device may include a projection that piercingly engages the obstructing member and the air passageway wall. The anchoring device may include a projection that piercingly engages the obstructing member and the air passageway wall. The projection may be releasable from the air passageway wall for removal of the anchoring device, and the step of removing the anchoring device includes releasing the projection. The obstructing member may form a one-way valve. A portion of the anchoring device may be collapsible.

Yet another embodiment provides an air passageway obstructing device having obstructing means for obstructing air flow within the air passageway, and anchoring means for anchoring the obstructing means within an air passageway by engaging the obstructing means and the air passageway, and the anchoring means being further releasable for removal of the obstructing means.

These and various other features as well as advantages which characterize the present invention will be apparent from a reading of the following detailed description and a review of the associated drawings.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify identical elements, and wherein:

FIG. 1 is a simplified sectional view of a thorax illustrating a healthy respiratory system;

FIG. 2 is a sectional view similar to FIG. 1, but illustrating a respiratory system suffering from COPD, and the execution of a first step in treating the COPD condition by reducing the size of a lung portion in accordance with the present invention;

FIG. 3 is perspective view, partially in section, and to an enlarged scale, illustrating an intermediate step in the treatment;

FIG. 4 illustrates an anchoring device being delivered through a catheter for placement in proximity to the obstructing member and deployment, in accordance with the invention;

FIG. 5 illustrates the obstructing device anchored in place within an air passageway by the anchoring device, in accordance with the invention;

FIG. 6 is a perspective view of an anchoring device, as the device would appear when fully deployed in an air passageway, in accordance with the present invention;

FIG. 7 is a perspective view of an intra-bronchial device comprising an obstructing member and the anchoring device of FIG. 6 anchored in an air passageway in accordance with the present invention;

FIG. 8 is a perspective view of an annular anchoring device as the device would appear when fully deployed in an air passageway, in accordance with the present invention;

FIG. 9 is a perspective view of an intra-bronchial device comprising an obstructing member and the annular anchoring device of FIG. 8 anchored in an air passageway, in accordance with the present invention; and

FIG. 10 is a plan view of the annular anchoring device of FIG. 8 engaged in the proximal end of an obstructive device, in accordance with the present invention.

### DETAILED DESCRIPTION

In the following detailed description of exemplary embodiments of the invention, reference is made to the accompanying drawings that form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined by the appended claims.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

Additionally, throughout the specification, claims, and drawings, the term “proximal” means nearest the trachea, and “distal” means nearest the bronchioles.

Briefly stated, an anchored intra-bronchial device is provided for placement in an air passageway of a patient to collapse or reduce ventilation to a lung portion associated with the air passageway. An obstructing member is first placed in the air passageway, and then an anchoring device is deployed which anchors the obstructing member in place. A further aspect of the invention provides removability of the intra-bronchial device by releasing the anchoring device for removal of the obstructing member.

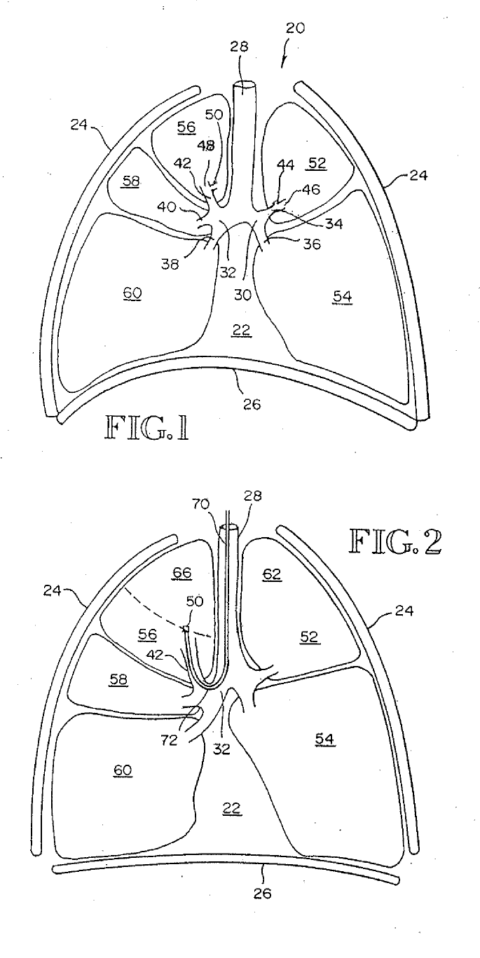

FIG. 1 is a sectional view of a healthy respiratory system. The respiratory system 20 resides within the thorax 22 that occupies a space defined by the chest wall 24 and the diaphragm 26.

The respiratory system 20 includes the trachea 28, the left mainstem bronchus 30, the right mainstem bronchus 32, the bronchial branches 34, 36, 38, 40, and 42 and sub-branches 44, 46, 48, and 50. The respiratory system 20 further includes left lung lobes 52 and 54 and right lung lobes 56, 58, and 60. Each bronchial branch and sub-branch communicates with a respective different portion of a lung lobe, either the entire lung lobe, a segment, or a portion thereof. As used herein, the term “air passageway” is meant to denote either bronchi or bronchioles, and typically means a bronchus branch or sub-branch that communicates with a corresponding individual lung lobe, segment, or lung lobe tissue portion to provide inhaled air thereto or conduct exhaled air therefrom.

Characteristic of a healthy respiratory system is the arched or inwardly arcuate diaphragm 26. As the individual inhales, the diaphragm 26 straightens to increase the volume of the thorax 22. This causes a negative pressure within the thorax. The negative pressure within the thorax in turn causes the lung lobes to fill with air. When the individual exhales, the diaphragm returns to its original arched condition to decrease the volume of the thorax. The decreased volume of the thorax causes a positive pressure within the thorax which in turn causes exhalation of the lung lobes.

In contrast to the healthy respiratory system of FIG. 1, FIG. 2 illustrates a respiratory system suffering from COPD. Here it may be seen that the lung lobes 52, 54, 56, 58, and 60 are enlarged and that the diaphragm 26 is not arched but substantially straight. Hence, this individual is incapable of breathing normally by moving diaphragm 28. Instead, in order to create the negative pressure in thorax 22 required for breathing, this individual must move the chest wall outwardly to increase the volume of the thorax. This results in inefficient breathing causing these individuals to breathe rapidly with shallow breaths.

It has been found that the apex portions 62 and 66 of the upper lung lobes 52 and 56, respectively, are most affected by COPD. Hence, bronchial sub-branch obstructing devices are generally employed for treating the apex 66 of the right, upper lung lobe 56. However, as will be appreciated by those skilled in the art, the present invention may be applied to any lung portion without departing from the present invention. As will be further appreciated by those skilled in the art, the present invention may be used with any type of obstructing member to provide an anchored obstructing device, which may be removed. The inventions disclosed and claimed in U.S. Pat. Nos. 6,258,100 and 6,293,951, both of which are incorporated herein by reference, provide an improved therapy for treating COPD by obstructing an air passageway using an intra-bronchial valve or plug. The present invention may be used with the apparatus, system, and methods of these patents as will be briefly described in conjunction with the disclosure of the preferred embodiments of the present invention.

The insertion of an obstructing member treats COPD by deriving the benefits of lung volume reduction surgery without the need of performing the surgery. The treatment contemplates permanent partial or complete collapse of a lung portion to reduce the volume of lung mass. This leaves extra volume within the thorax for the diaphragm to assume its arched state for acting upon the remaining healthier lung tissue. As previously mentioned, this should result in improved pulmonary function due to enhanced elastic recoil, correction of ventilation/perfusion mismatch, improved efficiency of respiratory musculature, and improved right ventricle filling. The present invention supports the use of intra-bronchial plugs to treat COPD by anchoring the obstructing member in the air passageway. The present invention further supports the use of intra-bronchial plugs by providing for their removal if necessary. Use of anchors can allow the obstructing member to be relatively loosely fitted against the air passageway wall, which may provide increased mucociliary transport of mucus and debris out of the collapsed lung portion.

FIG. 2 also illustrates a step in COPD treatment using an obstructing member using a bronchoscope or catheter. The invention disclosed herein is not limited to use with the particular method illustrated herein. Catheter 70 may be used alone to perform the insertion, may be extended from a bronchoscope, or used in conjunction with a bronchoscope. For purposes of this description, the insertion will be described with reference to only the catheter 70. Treatment is initiated by feeding a conduit or catheter 70 down the trachea 28, into the right mainstem bronchus 32, into the bronchial branch 42 and into and terminating within the sub-branch 50. The sub-branch 50 is the air passageway that communicates with the lung portion 66 to be treated, and is also referred to herein as air passageway 50. The catheter 70 is preferably formed of flexible material such as polyethylene. Also, the catheter 70 is preferably preformed with a bend 72 (or capable of bending) to assist the feeding of the catheter from the right mainstem bronchus 32 into the bronchial branch 42, or could be deformed to conform to different curvature and angles of a bronchial tree.

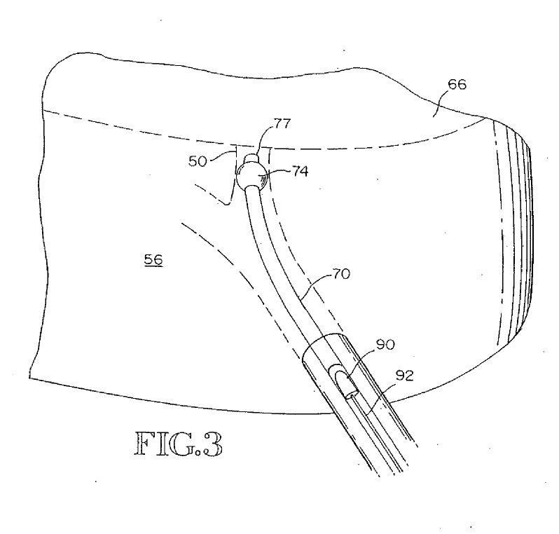

FIG. 3 illustrates a further step in a method for inserting an obstructing member 90 in a bronchial sub-branch using a catheter or a bronchoscope Catheter 70 may include an optional inflatable sealing member 74 for use with a vacuum to collapse lung portion 66 prior to insertion of obstructing member 90. The obstructing member 90 may be formed of resilient or collapsible material to enable the obstructing member 90 to be fed through the conduit 70 in a collapsed state. A stylet or biopsy forceps, hereafter referred to as a stylet 92, is used to push the obstructing member 90 to the end 77 of the catheter 70 for inserting the obstructing member 90 within the air passageway 50 adjacent to the lung portion 66 to be permanently collapsed. Optional sealing member 74 is withdrawn after obstructing member 90 is inserted.

A function of the intra-bronchial device disclosed and claimed in this specification, including the detailed description and the claims, is described in terms of collapsing a lung portion associated with an air passageway to reduce lung volume. In some lungs, a portion of a lung may receive air from collateral air passageways. Obstructing one of the collateral air passageways may reduce the volume of the lung portion associated with the air passageway, but not completely collapse the lung portion as that term may be generally understood. As used in the description and claims herein, the meaning of “collapse” includes both a complete collapse of a lung portion and a partial collapse of a lung portion.

Once deployed, the obstructing member precludes inhaled air from entering the lung portion to be collapsed. In accordance with the present invention, it is preferable that the obstructing member takes the form of a one-way valve. In addition to precluding inhaled air from entering the lung portion, the member further allows air within the lung portion to be exhaled. This results in more rapid collapse of the lung portion. In addition, anchoring obstructing members that preclude both inhaled and exhaled airflow are contemplated as within the scope of the invention.

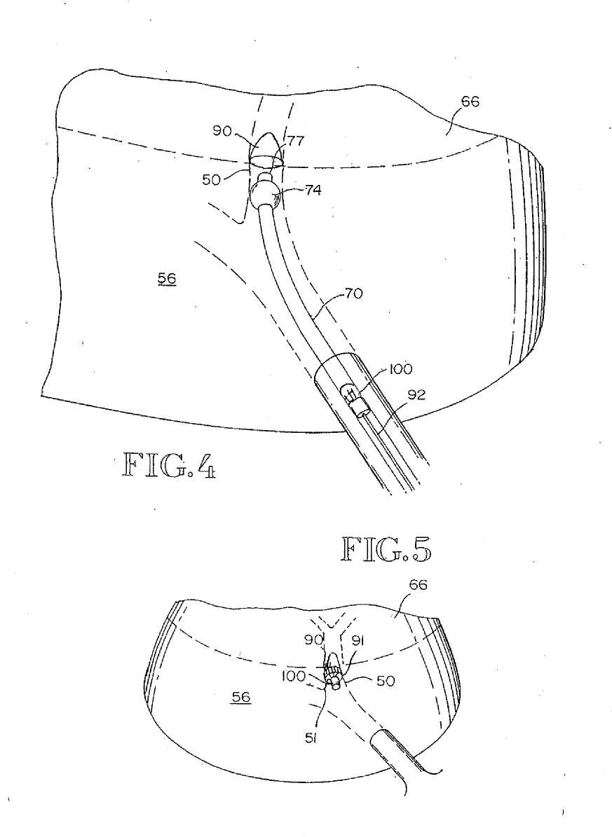

FIG. 4 illustrates an anchoring device being delivered through a catheter for placement in proximity to the obstructing member and deployment, in accordance with the invention. A previously compressed anchoring device 100 is pushed by stylet 92 to the end 77 of the catheter 70 for placement in proximity to the obstructing member 90. As anchoring device 100 is pushed from the catheter 70 into place and into proximity with the obstructing member 90, the resiliency of the anchor projections moves them peripherally. Anchoring device 100 is deployed by further advancing the stylet 92 to cause the projections of the anchoring device 100 to pierce the obstructing member 90 and the wall of the air passageway 50. This engagement by piercing anchors the obstructing member 90 in the air passageway 50.

FIG. 5 illustrates the obstructing device anchored in place within an air passageway by the anchoring device, in accordance with the invention. Obstructing member 90 has expanded upon placement in the air passageway 50 to loosely seal the air passageway 50. This causes the lung portion 66 to be maintained in a permanently collapsed state. The obstructing member 90 may be any shape suitable for accomplishing its purpose, and may be a solid member or a membrane. Anchoring device 100 has anchored obstructing member 90 in place by engaging both the obstructing member 90 and the wall of air passageway 50.

More specifically, the obstructing member 90 has an outer dimension 91, and when expanded, enables a contact zone with the air passageway inner dimension 51. This seals the air passageway upon placement of the obstructing member 90 in the air passageway 50 for maintaining the lung portion 66 in the collapsed state. The projections of the anchor 100 have engaged the obstructing member 90 and the wall of air passageway 50 by piercing into both. This engagement anchors obstructing member 90 against movement distally or proximally, such as might be caused by breathing, sneezing, coughing or gasping.

Alternatively, the lung portion 66 may be collapsed or reduced in volume using a vacuum prior to placement of obstructing member 90, or sealing the air passageway 50 with obstructing member 90 may collapse it. Over time, the air within the lung portion 66 will be absorbed by the body and result in the collapse of lung portion 66. Alternatively, obstructing member 90 may include the function of a one-way valve that allows air to escape from lung portion 66. Lung portion 66 will then collapse, and the valve will prevent air from being inhaled.

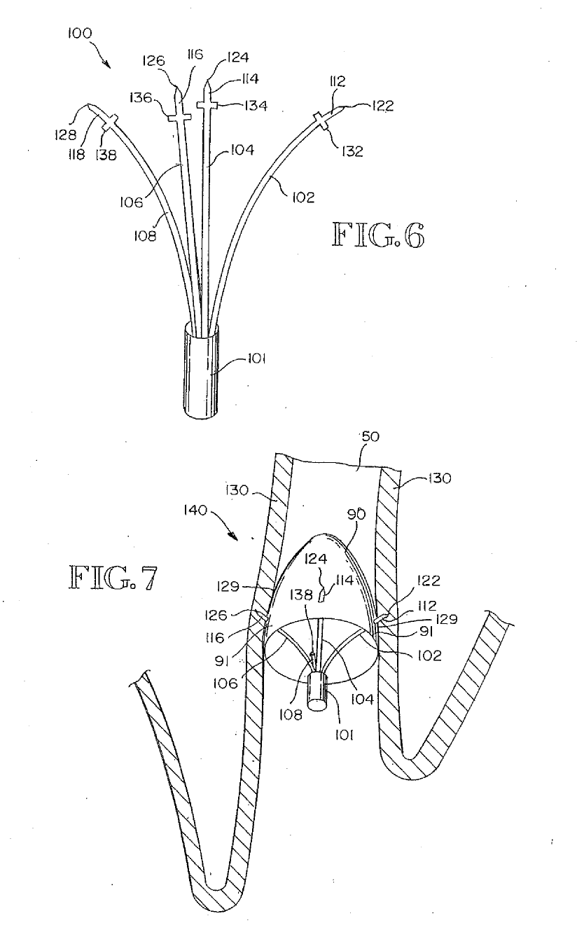

FIG. 6 is a perspective view of an anchoring device, as the device would appear when fully deployed in an air passageway, in accordance with the present invention. Anchoring device 100 includes a base 101, support members 102, 104, 106, and 108; projections 112, 114, 116, and 118; projection ends 122, 124, 126, and 128; and stops 132, 134, 136, and 138.

The base 101 of anchoring device 100 carries support members 102, 104, 106, and 108. The support members 102, 104, 106, and 108 carry projections 112, 114, 116, and 118, and projection ends 122, 124, 126, and 128, respectively. Base 101 is a tubular member, preferably hypodermic needle tubing. Support members 102, 104, 106, and 108, are coupled mechanically to base 101, such as by crimping, or by other methods such as adhesive or welding. Support members 102, 104, 106, and 108 are generally similar to each other. The support members are preferably formed of stainless steel, Nitinol, or other suitable material having a memory of its original shape, and resiliency to return the material to that shape. The support members and anchors may be formed by laser cutting a single tubular member, such as hypodermic needle tubing, lengthwise and bending the support members to the appropriate shape.

Projections 112, 114, 116, and 118 are portions of support members 102, 104, 106, and 108, respectively, and are at an end opposite to the end coupled to base 101. The support members and the projections are formed in a configuration that will result in the memory and resiliency of their material moving at least the projections proximally upon deployment to a position to engage the obstructing member and the air passageway wall by piercing. In this preferred embodiment, the configuration is a curve having a decreasing radius toward the projection ends, such that the projection ends will pierce the air passageway wall at an angle that provides sufficient shear resistance to anchor the obstructing member. The angle is a function of the design parameters of anchor device 100, and the more near perpendicular the angle is, the better the shear resistance will be. Projection ends 122, 124, 126, and 128 are shaped to promote piercing of an obstructing member and an air passageway wall. Stops 132, 134, 136, and 138, are shaped and dimensioned to limit the piercing by the projections, and generally consist of a widened area such as a shoulder between support members 102, 104, 106, and 108, and projections 112, 114, 116, and 118, respectively. The stops may be formed from the same material as the support member and its projection, or in an alternative embodiment, may be formed separately and coupled to the support member.

In an alternative embodiment, base 101, support members 102, 103, 104, 105, 106, and 108, projections 112, 114, 116, and 118, projection ends 122, 124, 126, and 128, and stops 132, 134, 136, and 138, may be formed by laser cutting a single tubular member lengthwise, and bending the support members and projections to a required shape. The tubular member is preferably hypodermic needle tubing, or may be stainless steel, Nitinol, or other suitable material having a memory of its original shape and resiliency to return the material to that shape.

FIG. 7 is a perspective view of an intra-bronchial device comprising an obstructing member and the anchoring device of FIG. 6 anchored in an air passageway, in accordance with the present invention. Intra-bronchial device 140 comprises obstructing member 90 and anchoring device 100. The obstructing member 90 illustrated includes a flexible membrane having an interior and exterior surface, open in the proximal direction, and may be formed of silicone, polyethylene, polyurethane, or other elastomeric material, for example. Obstructing member 90 may be carried on a support structure. In an alternative embodiment, obstructing member 90 may be a solid member.

FIG. 7 illustrates the obstructing member 90 anchored by the anchoring device 100. Projections 112, 114, 116, and 118 of anchoring device 100 engage obstructing member 90 and the air passageway wall 130 by piercing. This anchors the obstructing member 90 to the air passageway wall 130. The piercing is limited by stops 132, 134, 136, and 138. However, because of the perspective, only projections 112 and 116, and only stop 138 are visible.

Obstructing member 90 is collapsible for insertion into an internal lumen of a catheter. Obstructing member 90 is inserted into the catheter lumen, which is typically already placed in the air passageway 50 as generally illustrated in FIG. 3. Obstructing member 90 is advanced down the catheter lumen by a stylet into the air passageway 50 to where the obstructing member 90 is to be deployed. Once the point of deployment is reached, obstructing member 90 is released from the catheter and expands to assume its deployed shape as generally illustrated in FIG. 7. Upon deployment, obstructing member 90 forms a contact zone 129 with the wall 130 of the air passageway 50 to prevent air from being inhaled into the lung portion to collapse the lung portion. Obstructing member 90 may be loosely deployed such that it expands on inhalation to form a seal against a wall of the air passageway 130, and slightly contracts on exhalation to allow air and mucus transport from the collapsed lung portion. This provides a one-way valve function.

Anchoring device 100 is collapsed into a first position for insertion into the internal lumen of a catheter, which may be the same catheter that placed the obstructing member 90. Anchoring device 100 is inserted into the catheter lumen and advanced down the catheter lumen by pushing the stylet against base 101. Anchoring device 100 is advanced into the air passageway 50 to where it is to be deployed in proximity to obstructing member 90 as generally illustrated in FIGS. 4 and 5. Upon release from the catheter in proximity to obstructing member 90, projections 112, 114, 116, and 118 are urged peripherally by the memory and resiliency of the material of support members 102, 104, 106, and 108. Anchoring device 100 is further advanced by the stylet pushing against base 101, which imparts a force on the projections 122, 124, 126, and 128, and urges the projections to engage the obstructing member 90 and the air passageway wall 130 by piercing. The anchors pierce into and become embedded in the wall 130 of the air passageway 50, preferably without piercing through the wall 130. Stops 132, 134, 136, and 138 limit the piercing of the air passageway wall 130 by engaging obstructing member 90. This brings anchoring device 100 into its second position engaging the obstructing member 90 and the air passageway wall 130 to anchor obstructing member 90. In an alternative embodiment, the stops pierce the air passageway wall in the contact zone 129.

In another alternative embodiment, the anchoring device 100 is self-deploying. The memory and resiliency of the material of support members 102, 104, 106, and 108 provide sufficient urgency to force projections 122, 124, 126, and 128 to engage the obstructing member 90 and the air passageway wall 130 by piercing.

The preclusion of air from being inhaled into the lung portion may be terminated by eliminating the obstructing effect of intra-bronchial device 140. The preclusion of air by the embodiment illustrated in FIG. 7 may be eliminated by releasing projections 112, 114, 116, and 118 from the air passageway wall 130. The anchors may be released by inserting a catheter into air passageway 50 in proximity to anchor device 100. A retractor device, which may be biopsy forceps or other device capable of gripping a portion of anchor device 100, is inserted in the catheter. The forceps are used to engage a portion of the anchor device 100, preferably base 101, and draw it toward the catheter. The drawing action releases projections 112, 114, 116, and 118 from air passageway wall 130 and the obstructing member 90. The anchoring device 100 is drawn into the catheter with the forceps, causing the support members 102, 104, 106, and 108, and projections 112, 114, 116, and 118 to collapse into the first position. The collapsed anchoring device 100 now fully enters the catheter lumen for removal from the patient. The retractor device is then reinserted in the catheter. The forceps are used to engage obstructing member 90 and draw it toward the catheter. The drawing action releases obstructing member 90 from air passageway wall 130. The obstructing member 90 is then further drawn into the catheter with the forceps, causing it to collapse and fully enter the catheter lumen for removal from the patient.

FIG. 8 is a perspective view of an annular anchoring device, as the device would appear when fully deployed in an air passageway in accordance with the present invention. Annular anchoring device 150 includes annular member 162; periphery 164; aperture 152; projections 172, 174, 176, and 178; projection ends 182, 184, 186, and 188; and stops 192a-b, 194a-b, 196a-b, and 198a-b.

Annular member 162 has a periphery 164 and an aperture 152. Annular member 162 carries projections 172, 174, 176, and 178 on its periphery 164. Projection ends 182, 184, 186, and 188 are shaped to promote piercing of an obstructing member and an air passageway wall by the projections. Stops 192a-b, 194a-b, 196a-b, and 198a-b may be formed on the periphery 164 of annular member 162 adjacent to projections 172, 174, 176, and 178, respectively. The “a” stop and the “b” stop are disposed on opposite sides of a projection. Stops 192a-b, 194a-b, 196a-b, and 198a-b are shaped and dimensioned to limit the piercing of an obstructing member and an air passageway wall by the projections. In an alternative embodiment, the stops may form a shoulder completely around a perimeter of the projection.

Annular anchoring device 150 is made from stainless steel, Nitinol, or other suitable material having a memory of its original shape and resiliency to return the material to that shape. In an embodiment, annular anchoring device 150 is formed from a single piece of material, such as laser cutting, stamping, or other methods as are known to those in the art. Annular anchoring device 150 may have any cross-sectional shape compatible with its material and layout, which may be flat, elliptical, or rectangular. The number of projections, and the shape and configuration of the projection, may be selected as will provide sufficient engagement to anchor obstructing member 90.

In an alternative embodiment, the projections and their ends are arranged to frictionally engage without piercing. In a further alternative embodiment, the projections may be divided into sets, one set arranged to pierce and another set arranged not to pierce. One set of projections of this embodiment is further arranged to engage only the obstructing member 90 and the another set is arranged to engage only the air passageway wall 130.

In a preferred embodiment, anchoring device 150 is arranged to be balloon expandable into its fully deployed configuration illustrated in FIG. 8. In an alternative embodiment, anchoring device 150 is arranged to be centrally collapsible for delivery through a catheter, and then expanded to its fully deployed configuration by the force of its resiliency or by an external force.

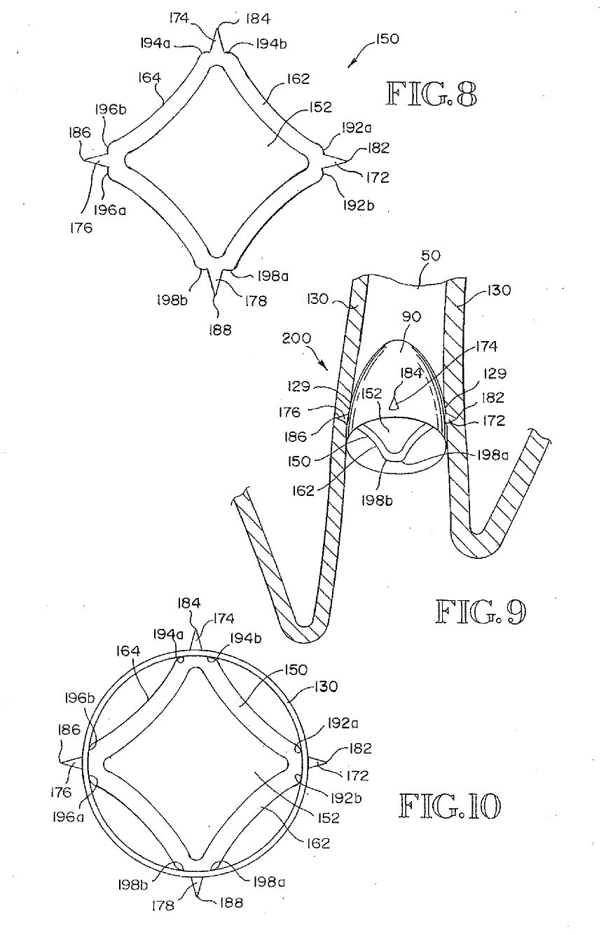

FIG. 9 is a perspective view of an intra-bronchial device comprising an obstructing member and the annular anchoring device of FIG. 8 anchored in an air passageway, in accordance with the present invention. Intra-bronchial device 200 comprises obstructing member 90 and annular anchoring device 150. FIG. 9 illustrates the obstructing member 90 anchored by the anchoring device 150. Projections 172, 174, 176, and 178 of anchoring device 150 engage obstructing member 90 and the air passageway wall 130 by piercing. This anchors the obstructing member 90 to the air passageway wall 130. The piercing is limited by stops 192a-b, 194a-b, 196a-b, and 198a-b. However, because of the perspective, projection 178 is not visible, and stops 192a-b, 194a-b, 196a-b are not visible.

Obstructing member 90 is placed in air passageway 50 in the manner described in conjunction with FIG. 7. In a preferred embodiment, anchoring device 150 is provided in a collapsed configuration, which is a first position, and is balloon expandable. In an alternative embodiment, anchoring device 150 may be collapsed into the first position by gripping opposed portions of periphery 164 with forceps, and drawing the portions toward each other. Anchoring device 150 in the first position is inserted into the internal lumen of a catheter, which may be the same catheter that placed the obstructing member 90. Anchoring device 150 is advanced down the catheter lumen placed into the air passageway 50 by pushing the stylet. Anchoring device 150 is advanced to where it is to be deployed in proximity to obstructing member 90 as generally illustrated in FIGS. 4 and 5. Anchoring device 150 is released from the catheter in proximity to obstructing member 90, such that when anchoring device is expanded, projections 172, 174, 176, and 178 move peripherally into a second position and engage obstructing member 90 and air passageway wall 130. In a preferred embodiment, the deployment includes expanding anchoring device 150 by a balloon catheter. The expansion of anchoring device 150 urges the projections 172, 174, 176, and 178 into engagement with the obstructing member 90 and the air passageway wall 130 by piercing, preferably without projecting through the wall 130. Stops 192a-b, 194a-b, 196a-b, and 198a-b limit the piercing of the air passageway wall 130 by engaging obstructing member 90.

In an alternative embodiment, the deployment includes expansion by the memory and resiliency of the material of anchoring device 150 urging the projections 172, 174, 176, and 178 to engage the obstructing member 90 and the air passageway wall 130. In a further alternative embodiment, the expansion may be provided or supplemented by a device deployed through the catheter that engages and expands aperture 152 to move anchoring device 150 into its deployed, or second position.

The preclusion of air from being inhaled into the lung portion may be terminated by eliminating the obstructing effect of intra-bronchial device 200. The preclusion of air by the embodiment illustrated in FIG. 9 may be eliminated by releasing projections 172, 174, 176, and 178 from the air passageway wall 130. The anchors may be released by inserting a catheter into air passageway 50 in proximity to anchor device 150. A retractor device, such as biopsy forceps, capable of gripping a portion of annular anchor device 150 is inserted in the catheter. The forceps are used to engage anchor device 150 and collapse it. Anchor device 150 can be collapsed by centrally moving opposing portions of the periphery 164 with the forceps to move anchor device 150 into the first position. The collapsing releases projections 172, 174, 176, and 178 from the air passageway wall 130 and the obstructing member 90. The forceps are used to draw anchoring device 150 into the catheter. The collapsed anchoring device 150 is fully drawn into the catheter lumen for removal from the patient. The retractor device is then reinserted in the catheter. The forceps are used to engage obstructing member 90 and draw it toward the catheter. The drawing action releases obstructing member 90 from air passageway wall 130. The obstructing member 90 is then further drawn into the catheter with the forceps, causing it to collapse and fully enter the catheter lumen for removal from the patient.

FIG. 10 is a plan view of the annular anchoring device of FIG. 8 engaged in the proximal end of an obstructive device, in accordance with the present invention. Annular anchoring device 150 is illustrated fully expanded and deployed into obstructing member 90. Projections 172, 174, 176, and 178 are illustrated having pierced through obstructing member 90, with the piercing limited by stops 192a-b, 194a-b, 196a-b, and 198a-b.

While particular embodiments of the present invention have been shown and described, modifications may be made. It is therefore intended in the appended claims to cover all such changes and modifications that fall within the true spirit and scope of the invention.

1. An intra-bronchial device comprising:
an obstructing member comprising a one-way valve having an umbrella shape that is configured to preclude fluid flow through the member; and
a barbed anchoring member provided separate of the obstructing member and configured to anchor the obstructing member in an air passageway, wherein the anchoring member is configured to be deployed separate of the obstructing member.

2. The intra-bronchial device of claim 1, wherein the anchoring member is releasable separate of the obstructing member for removal of the obstructing member.

3. The intra-bronchial device of claim 1, wherein the anchoring member comprises an original undistorted shape, and wherein the anchoring member is configured to be biased from a distorted shape to the original undistorted shape.

4. An intra-bronchial device comprising an obstructing member, said obstructing member comprising a resilient material, said obstructing member defining an outer dimension that, when said obstructing member is in an expanded state, is configured to contact an inner dimension of an air passageway, the intra-bronchial device further comprising a non-stent anchoring device that is separate of said obstructing member such that said obstructing member and said anchoring device can be inserted into the air passageway separate of each other, said anchoring device comprising a proximal base and distal ends, said distal ends being adapted to pierce said obstructing member and the air passageway when said anchoring device is deployed within said separately inserted obstructing member.

5. The intra-bronchial device of claim 4, wherein at least one of said distal ends of said anchoring device comprises a stop positioned proximally of said at least one distal end, said stop being configured to limit a length of said at least one distal end that pierces the air passageway.

6. The intra-bronchial device of claim 4, wherein said obstructing member assumes a generally umbrella-shaped configuration when deployed.

7. The intra-bronchial device of claim 6, wherein said generally umbrella-shaped configuration defines a convex recess that ends at a proximal end of said obstruction member and said anchoring device extends partway into said convex recess.

8. The intra-bronchial device of claim 7, wherein said anchoring device extends only partway into said convex recess.

9. The intra-bronchial device of claim 4, wherein said anchoring device comprises support members that are connected to said proximal base and that extend to said distal ends.

10. The intra-bronchial device of claim 9, wherein said support members are resilient.

11. The intra-bronchial device of claim 9, wherein said support members comprise define a curve having a decreasing radius toward said distal ends.

12. An intra-bronchial device comprising an obstructing member and an anchor device that are separate from each other until positioned in an air passageway, said obstructing member comprising a flexible membrane, said flexible membrane comprising an interior surface and an exterior surface, said flexible membrane defining a recess that is open in a proximal direction when positioned in the air passageway and said flexible membrane adapted to deploy separate of said anchor device, said anchor device extending at least partway into said recess, said anchor device comprising projections configured to engage said obstructing member and the air passageway and stops being positioned proximally of said projections.

13. The intra-bronchial device of claim 12, wherein said anchor device further comprises a base being connected to said projections and being positioned proximally of said projections.

14. The intra-bronchial device of claim 13, wherein said anchor device further comprises support members that extend between said stops and said base.

15. The intra-bronchial device of claim 12, wherein said anchor device comprises a generally annular member, said projections extending from said generally annular member.

16. The intra-bronchial device of claim 15, wherein said generally annular member defines an aperture.

17. The intra-bronchial device of claim 16, wherein said aperture is sized to accommodate a balloon such that said anchor device is balloon-expandable.

18. The intra-bronchial device of claim 12, wherein said projections are configured to pierce said obstructing member and the air passageway.

19. The intra-bronchial device of claim 12, wherein all of said projections are configured to engage the air passageway through the obstructing member.

20. An intra-bronchial device comprising an obstructing member and separately deployed means for anchoring said obstructing member in an air passageway, said obstructing member comprising a flexible membrane, said membrane comprising an interior surface and an exterior surface, said flexible membrane defining a recess that is open in a proximal direction when positioned in the air passageway, and at least a portion of said means for anchoring being positioned within said recess.

21. The intra-bronchial device of claim 20, wherein said means for anchoring is positioned fully within said recess.

22. The intra-bronchial device of claim 20, wherein said means for anchoring comprises a generally annular member.

23. The intra-bronchial device of claim 20, wherein said means for anchoring comprises a proximal base.

24. The intra-bronchial device of claim 23, wherein at least a portion of said proximal base is located outside of said recess.

25. The intra-bronchial device of claim 20, wherein said means for anchoring pierces said flexible membrane.
